(19) United States
(12) Patent Application Publication
(10) Pub. No.: US 2023/0356178 A1
(43) Pub. Date: Nov. 9, 2023

(54) ADSORBENT, METHOD FOR MANUFACTURING SAME, ADSORPTION SHEET, SEPARATION FILM, AND ARTIFICIAL DIALYSIS EQUIPMENT

(71) Applicant: Murata Manufacturing Co., Ltd., Nagaokakyo-shi (JP)

(72) Inventors: Yuusuke OGAWA, Nagaokakyo-shi (JP); Masashi KOYANAGI, Nagaokakyo-shi (JP); Yuki KIMURA, Nagaokakyo-shi (JP)

(21) Appl. No.: 18/348,527

(22) Filed: Jul. 7, 2023

Related U.S. Application Data
(63) Continuation of application No. PCT/JP2022/000037, filed on Jan. 4, 2022.

(30) Foreign Application Priority Data
Jan. 13, 2021 (JP) 2021-003541
Feb. 25, 2021 (JP) 2021-028821

(51) Int. Cl. B01J 20/04; B01J 20/28; B01J 20/02; B01J 20/20; B01J 20/30; B01J 20/32

(52) U.S. Cl. CPC B01J 20/04; B01J 20/28035; B01J 20/0211; B01J 20/0288; B01J 20/20; B01J 20/3071; B01J 20/3021; B01J 20/3078; B01J 20/3204

(57) ABSTRACT

An adsorbent that includes: particles of a layered material including one or plural layers; and one or more metal atoms selected from Al, Mg, Ca, Ba, Fe, Zn, Mn, or Cu. The one or plural layers include a layer body represented by: MₘXₙ wherein M is at least one metal of Group 3, 4, 5, 6, or 7, X is a carbon atom, a nitrogen atom, or a combination thereof, n is 1 to 4, and m is more than n and 5 or less. A modifier or terminal T exists on a surface of the layer body, T is at least one of a hydroxyl group, a fluorine atom, a chlorine atom, an oxygen atom, or a hydrogen atom, and the M of the layer body is bonded to at least one of a chlorine atom, a phosphorus atom, an iodine atom, or a sulfur atom.

### CROS REFERENCE TO RELATED APPLICATIONS

The present application is a continuation of International application No. PCT/JP2022/000037, filed Jan. 4, 2022, which claims priority to Japanese Patent Application No. 2021-003541, filed Jan. 13, 2021, and Japanese Patent Application No. 2021-028821, filed Feb. 25, 2021, the entire contents of each of which are incorporated herein by reference.

### TECHNICAL FIELD

The present disclosure relates to an adsorbent, a method for manufacturing the same, an adsorption sheet, a separation film, and an artificial dialysis equipment.

### BACKGROUND ART

In recent years, MXene has been attracting attention as a new material. MXene is a type of so-called two-dimensional material, and as will be described later, is a layered material in the form of one or plural layers. In general, MXene is in the form of particles (which may also be referred to as MXene particles and can include powders, flakes, nanosheets, and the like) of such a layered material.

Currently, various studies are being conducted toward the application of MXene to various applications such as electronic devices and medical devices. For example, Non-patent Document 1 and Non-patent Document 2 disclose that by ion-exchanging Li⁺ with Mg²⁺ and Ca²⁺, Mg²⁺, and Ca²⁺ are intercalated between layers of MXene. In addition, Non-patent Document 2 discloses performing intercalation of Na and K and an electrode using MXene. Furthermore, Patent Document 1 discloses a method of intercalating Mg²⁺ and Ca²⁺ by adding MgF₂ and CaF₂ during etching. For the above application, it is required to enhance the adsorption performance of MXene. In addition, as applications other than the electrode, Non-patent Document 3 discloses that MXene is used for urea removal in dialysis.
Patent Document 1: U.S. Pat. No. 10,683,208 B2
Non-patent Document 1: Michael Ghidiu et al., Ion-Exchange and Cation Solvation Reactions in Ti3C2 MXene, Chem. Mater. 2016, 28, 3507-3514
Non-patent Document 2: Shuo Li et al., Intercalation of Metal Ions into Ti3C2Tx MXene Electrodes for High-Areal-Capacitance Microsupercapacitors with Neutral Multivalent Electrolytes, Adv. Funct. Mater. 2020, 30, 2003721
Non-patent Document 3: Fayan Meng et al., MXene Sorbents for Removal of Urea from Dialysate: A Step toward the Wearable Artificial Kidney, ACS Nano 2018, 12, 10518-10528

### SUMMARY OF THE INVENTION

As disclosed in Non-patent Document 3, in recent years, studies using MXene as an adsorbent have also been conducted, but it is difficult to say that known techniques have sufficient adsorption performance. The present disclosure has been made in view of the above circumstances, and an object of the present disclosure is to provide an adsorbent having excellent adsorption performance.

According to an aspect of the present invention, there is provided an adsorbent comprising:

particles of a layered material including one or plural layers; and

one or more metal atoms selected from the group consisting of Al, Mg, Ca, Ba, Fe, Zn, Mn, or Cu,

wherein the one or plural layers include a layer body represented by:

MₘXₙ

wherein M is at least one metal of Group 3, 4, 5, 6, or 7,
X is a carbon atom, a nitrogen atom, or a combination thereof,
n is 1 to 4, and
m is more than n and 5 or less, and

a modifier or terminal T exists on a surface of the layer body, wherein T is at least one selected from the group consisting of a hydroxyl group, a fluorine atom, a chlorine atom, an oxygen atom, or a hydrogen atom, and

wherein the M of the layer body is bonded to at least one selected from the group consisting of a chlorine atom, a phosphorus atom, an iodine atom, or a sulfur atom.

According to another aspect of the present invention, there is provided a method for manufacturing an adsorbent, the method comprising:

(a) preparing a precursor represented by:

MₘAXₙ
wherein M is at least one metal of Group 3, 4, 5, 6, or 7,
X is a carbon atom, a nitrogen atom, or a combination thereof,
A is at least one element of Group 12, 13, 14, 15, or 16,
n is 1 to 4, and
m is more than n and 5 or less;

(b) performing etching treatment of removing at least a part of A atoms from the precursor by using an etching solution containing one or more of HCl, H₃PO₄, HI, and H₂SO₄ to obtain an etched product;

(c) acid-washing the etched product to obtain an acid-washed product;

(d) water-washing the acid-washed product to adjust the pH of the acid-washed product to obtain a water-washed product;

(e) performing metal atom intercalation treatment including a step of mixing the water-washed product with a compound containing one or more metal atoms selected from the group consisting of Al, Mg, Ca, Ba, Fe, Zn, Mn, or Cu to obtain a metal atom intercalated product; and

(f) washing the metal atom intercalated product with water to obtain an adsorbent.

According to the present disclosure, an adsorbent is formed of a predetermined layered material (also referred to as “MXene” in the present specification), contains one or more of metal atoms selected from the group consisting of Al, Mg, Ca, Ba, Fe, Zn, Mn, and Cu, or M in MXene and at least one selected from the group consisting of a chlorine atom, a phosphorus atom, an iodine atom, or a sulfur atom are bonded to each other, thereby providing an adsorbent containing MXene and having excellent adsorption performance.

Further, according to the present invention, an adsorbent which contains the metal atom and in which M in MXene is bonded to at least one selected from the group consisting of a chlorine atom, a phosphorus atom, an iodine atom, and or a sulfur atom, and which is excellent in adsorption performance of, for example, a polar organic compound can be manufactured. The adsorbent can be manufactured by the method including (a) preparing a predetermined precursor; (b) performing etching treatment of removing at least a part of A atoms from the precursor by using a predetermined etching solution; (c) acid-washing an etched product obtained by the etching treatment; (d) water-washing an acid-washed product obtained by the acid-washing to adjust the pH of the acid-washed product; (e) performing metal atom intercalation treatment including a step of mixing a water-washed product obtained by the water-washing with a compound containing one or more metal atoms selected from the group consisting of Al, Mg, Ca, Ba, Fe, Zn, Mn, or Cu; and (f) washing a metal atom intercalated product obtained by the metal atom intercalation treatment with water.

### BRIEF DESCRIPTION OF THE DRAWINGS

FIG. 1 shows a schematic cross-sectional view of MXene which is a layered material usable for an adsorbent of the present invention, in which FIG. 1(a) illustrates single-layer MXene, and FIG. 1(b) illustrates multilayer (exemplarily two-layer) MXene.

FIG. 2 shows an interlayer distance in the adsorbent according to the present invention.

FIG. 3 shows a schematic illustration of artificial dialysis equipment using the adsorbent according to the present invention.

FIG. 4 shows X-ray diffraction measurement results in examples.

### DETAILED DESCRIPTION

### First Embodiment: Adsorbent

Hereinafter, an adsorbent in one embodiment of the present invention will be described in detail, but the present invention is not limited to such an embodiment.

An adsorbent in the present embodiment comprises:

particles of a layered material including one or plural layers; and

one or more metal atoms selected from the group consisting of Al, Mg, Ca, Ba, Fe, Zn, Mn, or Cu,

wherein the one or plural layers include a layer body represented by:

MₘXₙ
wherein M is at least one metal of Group 3, 4, 5, 6, or 7,
X is a carbon atom, a nitrogen atom, or a combination thereof,
n is 1 to 4, and
m is more than n and 5 or less, and

a modifier or terminal T exists on a surface of the layer body, wherein T is at least one selected from the group consisting of a hydroxyl group, a fluorine atom, a chlorine atom, an oxygen atom, or a hydrogen atom, and

wherein M of the layer body is bonded to at least one selected from the group consisting of a chlorine atom, a phosphorus atom, an iodine atom, or a sulfur atom.

The layered material can be understood as a layered compound and is also denoted by “MₘXₙTₛ”, in which s is an optional number, and in the related art, x or z may be used instead of s. Typically, n can be 1, 2, 3, or 4, but is not limited thereto.

In the above formula of MXene, M is preferably at least one selected from the group consisting of Ti, Zr, Hf, V, Nb, Ta, Cr, Mo, or Mn, and more preferably at least one selected from the group consisting of Ti, V, Cr, or Mo.

MXenes whose above formula MₘXₙ is expressed as below are known:

Sc₂C, Ti₂C, Ti₂N, Zr₂C, Zr₂N, Hf₂C, Hf₂N, V₂C, V₂N, Nb₂C, Ta₂C, Cr₂C, Cr₂N, Mo₂C, Mo_{1.3}C, Cr_{1.3}C, (Ti,V)₂C, (Ti,Nb)₂C, W₂C, W_{1.3}C, Mo₂N, Nb_{1.3}C, Mo_{1.3}Y_{0.6}C (in the above formula, “1.3” and “0.6” mean about 1.3 (= 4/3) and about 0.6 (=⅔), respectively),

Ti₃C₂, Ti₃N₂, Ti₃(CN), Zr₃C₂, (Ti,V)₃C₂, (Ti₂Nb)C₂, (Ti₂Ta)C₂, (Ti₂Mn)C₂, Hf₃C₂, (Hf₂V)C₂, (Hf₂Mn)C₂, (V₂Ti)C₂, (Cr₂Ti)C₂, (Cr₂V)C₂, (Cr₂Nb)C₂, (Cr₂Ta)C₂, (Mo₂Sc)C₂, (Mo₂Ti)C₂, (Mo₂Zr)C₂, (Mo₂Hf)C₂, (Mo₂V)C₂, (Mo₂Nb)C₂, (Mo₂Ta)C₂, (W₂Ti)C₂, (W₂Zr)C₂, (W₂Hf)C₂,

Ti₄N₃, V₄C₃, Nb₄C₃, Ta₄C₃, (Ti,Nb)₄C₃, (Nb,Zr)₄C₃, (Ti₂Nb₂)C₃, (Ti₂Ta₂)C₃, (V₂Ti₂)C₃, (V₂Nb₂)C₃, (V₂Ta₂)C₃, (Nb₂Ta₂)C₃, (Cr₂Ti₂)C₃, (Cr₂V₂)C₃, (Cr₂Nb₂)C₃, (Cr₂Ta₂)C₃, (Mo₂Ti₂)C₃, (Mo₂Zr₂)C₃, (Mo₂Hf₂)C₃, (Mo₂V₂)C₃, (Mo₂Nb₂)C₃, (Mo₂Ta₂)C₃, (W₂Ti₂)C₃, (W₂Zr₂)C₃, (W₂Hf₂)C₃, (Mo_{2.7}V_{1.3})C₃ (in the above formula, “2.7” and “1.3” mean about 2.7 (=8/3) and about 1.3 (= 4/3), respectively).

Typically in the above formula, M can be titanium or vanadium and X can be a carbon atom or a nitrogen atom. For example, the MAX phase is Ti₃AlC₂ and MXene is Ti₃C₂Tₛ (in other words, M is Ti, X is C, n is 2, and m is 3).

It is noted that, in the present invention, MXene may contain remaining A atoms at a relatively small amount, for example, at 10 mass % or less with respect to the original amount of A atoms. The remaining amount of A atoms can be preferably 8 mass % or less, and more preferably 6 mass % or less. However, even if the residual amount of A atoms exceeds 10 mass %, there may be no problem depending on the application and use conditions of the adsorbent.

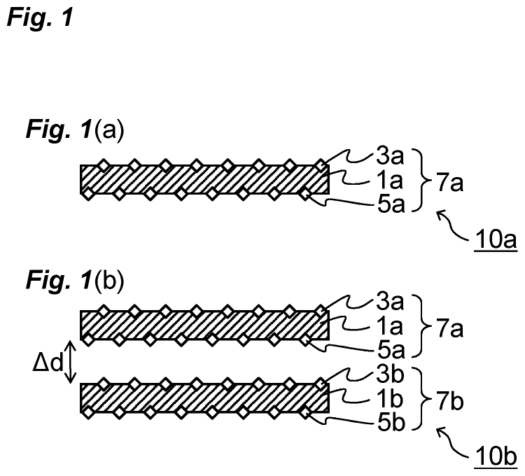

Hereinafter, MXene particles corresponding to the skeleton of the adsorbent according to the present embodiment will be described with reference to FIG. 1. FIG. 1 does not illustrate that a specific metal element is contained, and that M in the layer body is bonded to at least one selected from the group consisting of a chlorine atom, a phosphorus atom, an iodine atom, or a sulfur atom.

The adsorbent of the present embodiment is an aggregate containing one layer of MXene 10a (single layer MXene) schematically illustrated in FIG. 1(a). More specifically, MXene 10a is an MXene layer 7a having layer body (MₘXₙ layer) la represented by MₘXₙ, and modifier or terminals T3a and 5a existing on the surface (more specifically, at least one of two surfaces facing each other in each layer) of the layer body 1a. Therefore, the MXene layer 7a is also represented as “MₘXₙTₛ”, and s is an optional number.

The adsorbent of the present embodiment may include one layer and plural layers. Examples of the MXene (multilayer MXene) of the plural layers include, but are not limited to, two layers of MXene 10b as schematically illustrated in FIG. 1(b). 1b, 3b, 5b, and 7b in FIG. 1(b) are the same as 1a, 3a, 5a, and 7a in FIG. 1(a) described above. Two adjacent MXene layers (for example, 7a and 7b) of the multilayer MXene do not necessarily have to be completely separated from each other, and may be partially in contact with each other. The MXene 10a may be a mixture of the single-layer MXene 10a and the multilayer MXene 10b, in which the multilayer MXene 10b is individually separated and exists as one layer and the unseparated multilayer MXene 10b remains. The adsorbent of the present embodiment is preferably formed of particles of a layered material including plural layers, that is, multilayer MXene. By being formed of particles of a layered material including plural layers, a large amount of substance to be adsorbed can be adsorbed between layers of the plural layers, and adsorption performance can be improved.

Although the present embodiment is not limited, the thickness of each layer of MXene (which corresponds to the MXene layers 7a and 7b) is, for example, 0.8 nm to 5 nm, particularly 0.8 nm to 3 nm (which may mainly vary depending on the number of M atom layers included in each layer). For the individual laminates of the multilayer MXene that can be included, the interlayer distance (alternatively, a void dimension which is indicated by Ad in FIG. 1(b)) is, for example, 0.8 nm to 10 nm, particularly 0.8 nm to 5 nm, and more particularly about 1 nm, and the total number of layers can be 2 to 20,000.

The adsorbent of the present embodiment contains one or more metal atoms selected from the group consisting of Al, Mg, Ca, Ba, Fe, Zn, Mn, or Cu. In order to distinguish such a metal atom from a metal atom constituting MXene, the metal atom may be referred to as a “specific metal atom”, and the metal atom containing particles of a layered material and the specific metal atom may be referred to as a “specific metal atom-containing MXene” to distinguish from MXene not containing the specific metal atom.

The specific metal atom can be derived from an intercalator used for intercalation of the specific metal atom. The specific metal atom is preferably present between layers of MXene by intercalation of the specific metal atom. The specific metal atom may be in a state of a metal ion in MXene. That is, the metal ions can exist between layers of MXene as divalent metal ions. When the specific metal atom is present between the MXene layers and exerts the effect of a pillar supporting a wide range of layers, the substance to be adsorbed is easily inserted between the layers of MXene, and as a result, a large amount of the substance to be adsorbed can be adsorbed, and adsorption performance can be enhanced, which is preferable. For example, when the substance to be adsorbed is urea, the adsorbent has high adsorption characteristics of urea, and is excellent as a material for artificial dialysis. Furthermore, for example, when the substance to be adsorbed is a dye represented by methylene blue or the like, it is excellent as an adsorbent for removing the dye from industrial water, for example. In addition, the specific metal atoms such as Mg and Ca are present between the layers of MXene, and the layers of MXene are widened, so that impurities between the layers of MXene, for example, an acidic substance used at the time of manufacturing, and the like are easily removed at the manufacturing stage, and it is possible to suppress a pH change of the solution due to the acidic substance in the adsorbent when the obtained adsorbent comes into contact with the solution.

It is considered that by intercalating the specific metal atoms at the time of manufacturing the adsorbent, the specific metal atoms are inserted between the layers of MXene, and the distance between the layers is increased, so that the distance between the layers becomes more appropriate with respect to the size of the substance to be adsorbed, and the adsorption performance is improved. The specific metal atom is an element having a charge of 2 or more and capable of forming a water-soluble compound.

A content of the specific metal atom (the total content in the case of two or more kinds) may be 0.001 mass % to 3.0 mass %.

The specific metal atom preferably contains one or more selected from the group consisting of Mg, Ca, Fe, Zn, or Mn in consideration of biocompatibility. The specific metal atom is more preferably formed of one or more selected from the group consisting of Mg, Ca, Fe, Zn, or Mn. The specific metal atom more preferably contains one or more of Mg and Ca, for example, from the viewpoint of further improving the biocompatibility. The specific metal atom is particularly preferably Mg and/or Ca.

A total content of one or more of Mg and Ca in the specific metal atom is preferably 0.001 mass % to 1.5 mass %. From the viewpoint of further enhancing the biocompatibility, it is preferable that the number of specific metal atoms is smaller.

For example, as will be described later, in the case of an adsorbent which does not contain Li and contains one or more of Mg and Ca as the specific metal atoms, Mg and Ca exist as ions, and thus the biocompatibility is enhanced, and Mg²⁺ and Ca²⁺ preferably increase the interlayer distance, and as a result, an interlayer distance appropriate for the size of urea molecules is obtained, so that urea easily enters between MXene layers.

In the adsorbent of the present embodiment, M in the layer body is bonded to at least one selected from the group consisting of a chlorine atom, a phosphorus atom, an iodine atom, or a sulfur atom. The chlorine atom, the phosphorus atom, the iodine atom, and the sulfur atom can be derived from HCl (hydrochloric acid), H₃PO₄ (phosphoric acid), HI (hydrogen iodide), and H₂SO₄ (sulfuric acid) contained in the etching solution used at the time of etching the MAX phase, which is a precursor of MXene. That is, the chlorine atom in the adsorbent of the present embodiment is preferably Cl⁻ bonded to M of the layer body, the phosphorus atom is preferably a phosphorus atom constituting PO₄³⁻ bonded to M of the layer body, and the iodine atom in the adsorbent of the present embodiment is preferably I bonded to M of the layer body. In addition, the sulfur atom in the adsorbent of the present embodiment is preferably a sulfur atom constituting SO₄²⁻ bonded to M of the layer body.

(Li Content of Adsorbent)

In the adsorbent of the present embodiment, for example, a Li content is preferably equal to or less than the quantitative limit, for example, the Li content is preferably 0.0001 mass % or less (including 0 mass %). Since the Li content of the adsorbent is suppressed within the above range, the adsorbent of the present embodiment can be adopted for applications requiring the biocompatibility, such as a separation film in an artificial dialysis equipment. The Li content can be measured by, for example, ICP-AES using inductively coupled plasma emission spectrometry.

(Interlayer Distance of Adsorbent)

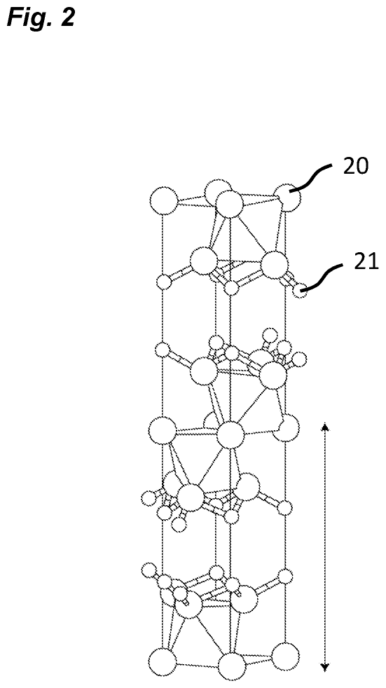

In the adsorbent of the present embodiment, as described above, it is considered that the specific metal atom is preferably inserted between the layers of MXene to expand the layers. When MₘXₙ is Ti₃C₂O₂ (O-term) represented by Ti₃C₂, the crystal structure is as schematically illustrated in FIG. 2 (in FIG. 2, reference numeral 20 denotes a titanium atom, reference numeral 21 denotes an oxygen atom, and other constituent atoms are not shown), and it is considered that the distance between the layers indicated by bidirectional arrows in FIG. 2 has increased. The above distance can be determined by the position of a low-angle peak of 10°(deg) or less corresponding to the (002) plane of MXene in an XRD profile obtained by X-ray diffraction measurement. The lower the angle of the peak in the XRD profile is, the wider the interlayer distance is. In the adsorbent in the present embodiment, the peak of the (002) plane obtained by X-ray diffraction measurement is preferably less than 8.0°. The peak is more preferably 7.0° or less. The lower limit of the peak position is about 5.0°. The peak refers to a peak top. The X-ray diffraction measurement may be performed under the conditions shown in examples to be described later.

When the adsorbent of the present embodiment has MXene in which MₘXₙ is represented by Ti₃C₂ and the specific metal atom, the interlayer distance obtained from the result of the XRD can be, for example, 12.0 Å or more, preferably 12.5 Å or more, and more preferably 13.0 Å or more, and the upper limit of the interlayer distance can be, for example, approximately 17.5 Å. It is considered that since the distance between the layers was increased, as a result, the distance between the layers has become an appropriate value with respect to the size of the urea molecule, and the adsorption performance was improved. In particular, since the interlayer distance in the above range is a size suitable for adsorption of uremic toxins, particularly urea, which needs to be removed by, for example, artificial dialysis, the adsorbent of the present embodiment is suitable for adsorption of the urea.

(Adsorbent Formed of Composite Material)

The adsorbent of the present embodiment may further include one or more materials of ceramic, metal, and a resin material. For example, as described in the examples below, when the adsorbent of the present embodiment is used for urea adsorption in artificial dialysis, by using a composite material (composite) of the specific metal atom-containing MXene according to the present embodiment and one or more materials among ceramic, metal, and a resin material, an adsorbent that stably exhibits adsorption performance, for example, adsorption performance of urea can be realized.

Examples of the ceramic include metal oxides such as silica, alumina, zirconia, titania, magnesia, cerium oxide, zinc oxide, barium titanate-based, hexaferrite, and mullite, and non-oxide ceramics such as silicon nitride, titanium nitride, aluminum nitride, silicon carbide, titanium carbide, tungsten carbide, boron carbide, and titanium boride. Examples of the metal include iron, titanium, magnesium, aluminum, and alloys based on these metals.

Examples of the resin material (polymer) include cellulose-based resins and synthetic polymer-based resins. Examples of the polymer include a hydrophilic polymer (includes a polymer that hydrophilicity is exhibited by mixing a hydrophilic auxiliary agent in a hydrophobic polymer, or hydrophilization treatment is performed on a surface of a hydrophobic polymer or the like), and the hydrophilic polymer more preferably includes one or more selected from the group consisting of polysulfone, cellulose acetate, regenerated cellulose, polyether sulfone, water-soluble polyurethane, polyvinyl alcohol, sodium alginate, an acrylic acid-based water-soluble polymer, polyacrylamide, polyaniline sulfonic acid, or nylon.

As the hydrophilic polymer, for example, a hydrophilic polymer having a polar group is preferably used, in which the polar group is a group that forms a hydrogen bond with a modifier or terminal T of the layer. As the polymer, for example, one or more polymers selected from the group consisting of water-soluble polyurethane, polyvinyl alcohol, sodium alginate, an acrylic acid-based water-soluble polymer, polyacrylamide, polyaniline sulfonic acid, or nylon are preferably used. Among these, one or more polymers selected from the group consisting of water-soluble polyurethane, polyvinyl alcohol, or sodium alginate are more preferable, and water-soluble polyurethane is still more preferable.

When the adsorbent formed of the composite material is used, for example, for a living body, examples of the polymer constituting the composite material include a polymer used for hemodialysis and hemofiltration. Specific examples thereof include polymethyl methacrylate, polyacrylonitrile, cellulose, cellulose acetate, polysulfone, polyvinyl alcohol, and a vinyl alcohol copolymer such as a copolymer of polyvinyl alcohol and ethylene. Preferably, one or more of polysulfone, polymethyl methacrylate, and cellulose acetate are used. More preferably, polysulfone or polymethyl methacrylate is used.

The ratio of the polymer contained in the composite material can be appropriately set according to the application. For example, the proportion of the polymer is more than 0 vol %, and can be, for example, 80 vol % or less, further 50 vol % or less, further 30 vol % or less, further 10 vol % or less, and even further 5 vol % or less in terms of the proportion in the adsorbent (when dried).

A method for manufacturing the adsorbent formed of the composite material is not particularly limited. When the adsorbent of the present embodiment contains a polymer and is an adsorbent having a sheet-like form, for example, as described in the examples below, a coating film can be formed by mixing the specific metal atom-containing MXene and a polymer.

First, a specific metal atom-containing MXene aqueous dispersion in which particles formed of the specific metal atom-containing MXene are present in a dispersion medium, a specific metal atom-containing MXene organic solvent dispersion, or a specific metal atom-containing MXene powder may be mixed with a polymer. The dispersion medium of the specific metal atom-containing MXene aqueous dispersion is typically water, and in some cases, other liquid substances may be contained in a relatively small amount (for example, 30 mass % or less, and preferably 20 mass % or less based on the whole mass) in addition to water.

The stirring of the specific metal atom-containing MXene particles and the polymer can be performed using a dispersing device such as a homogenizer, a propeller stirrer, a thin film swirling stirrer, a planetary mixer, a mechanical shaker, or a vortex mixer.

A slurry which is a mixture of the specific metal atom-containing MXene particles and the polymer may be applied to a substrate (for example, a substrate), but the application method is not limited. Examples of the coating method include a spray coating method in which spray coating is performed using a nozzle such as a one-fluid nozzle, a two-fluid nozzle, or an air brush, a slit coating method using a table coater, a comma coater, or a bar coater, a screen printing method, a metal mask printing method, a spin coating, and coating methods by immersion, or dropping.

The coating and drying may be repeated a plurality of times as necessary until a film having a desired thickness is obtained. The drying and curing may be performed, for example, at a temperature of 400° C. or lower using a normal pressure oven or a vacuum oven.

When the adsorbent of the present embodiment is a composite material containing a ceramic or a metal, examples of a method for manufacturing the adsorbent include a method in which particulate specific metal atom-containing MXene and particulate ceramic or metal are mixed and heated at a low temperature at which the composition of the specific metal atom-containing MXene can be maintained to form an adsorbent.

(Shape of Adsorbent)

The shape of the adsorbent of the present embodiment is not limited. The shape of the adsorbent may be a shape having a thickness, a rectangular parallelepiped, a sphere, a polygonal body, or the like other than a case of having a sheet-like form such as the film.

(Adsorption Sheet)

Preferred embodiments of the adsorbent of the present embodiment include an adsorption sheet. In addition to the adsorbent of the present embodiment, that is, the adsorption sheet formed of the specific metal element-containing MXene or the composite material containing the specific metal element, an adsorption sheet obtained by forming the adsorbent of the present embodiment on a substrate surface formed of one or more materials of ceramic, metal, and a resin material may be used. As the ceramic, metal, and resin material, the materials exemplified in the description of the above composite material can be used. Among them, an adsorption sheet obtained by forming the adsorbent of the present embodiment on a substrate formed of a resin material, preferably the above polymer, is preferable. In the aspect of the adsorbent of the present embodiment on the substrate, the adsorbent may be formed on the entire surface of the substrate by, for example, application or the like, or may be formed on at least a part of the substrate. As the forming method of the adsorbent on the substrate, for example, commonly used coating methods such as immersion, brush, roller, roll coater, air spray, airless spray, curtain flow coater, roller curtain coater, die coater, and electrostatic coating can be used. The thickness of the adsorption sheet and the thickness of the substrate can be appropriately set according to the application.

(Application of Adsorbent)

One application of the adsorbent of the present embodiment is to use the adsorbent for adsorption of a polar organic compound. The polar organic compound is a general term for organic compounds having polarity, and refers to a compound having a polar group such as an OH group, an NO₂ group, an NH group, an NH₂ group, or a COOH group, and in which a hydrogen atom in a water molecule and these polar groups can form a hydrogen bond when mixed with water. Examples of adsorption targets, among the polar organic compounds, include polar solvents such as alcohols having a hydroxyl group, compounds having an amino group, ammonia, and the like. The adsorbent of the present embodiment may be used for adsorbing a compound having one or more of a hydroxyl group and an amino group, and ammonia. Examples of the compound having a hydroxyl group among the compounds having the hydroxyl group and one or more amino groups include monohydric alcohols having 1 to 22 carbon atoms; polyhydric phenol; polyhydric alcohol such as ethylene glycol, propylene glycol, and glycerin; alkanolamines such as triethanolamine; and sugars such as xylose and glucose. Examples of the compound having an amino group include monoamines such as methylamine and dimethylamine; diamine such as ethylenediamine; polyamines such as diethylenetriamine; aromatic amines such as aniline; amino acids such as valine and leucine, urea, uric acid, urate, and creatinine. Examples of the compound having a hydroxyl group and an amino group include ethanolamine and diethanolamine.

The adsorbent of the present embodiment is preferably used for adsorbing uremic toxins including, for example, urea, uric acid, creatinine, and the like. The adsorbent of the present embodiment can be optimally used particularly for adsorbing urea.

The adsorbent of the present embodiment can be used for adsorbing and removing waste products such as urea in hemodialysis, hemofiltration, hemodiafiltration, peritoneal dialysis, and the like. In addition, the adsorbent of the present embodiment can be used in an artificial dialysis equipment for performing the hemodialysis, hemofiltration, hemodiafiltration, peritoneal dialysis, and the like.

The artificial dialysis equipment is classified into, for example, a hemodialysis equipment and a peritoneal dialysis equipment, and the hemodialysis equipment is divided into a one-pass type (single-pass type) and a circulation type. Further, circulation types include equipment of REDY system (recirculating dialysate system) and other systems. The artificial dialysis equipment is also divided by a method for removing urea without coming into contact with blood by a cross flow of blood from a patient and a dialysate and a method for directly filtering blood. In addition, the peritoneal dialysis equipment is mainly of one-pass type. The adsorbent of the present embodiment can be used for both of the hemodialysis and the peritoneal dialysis, and can be used as an adsorption film, a separation film, an adsorbent cartridge, or the like in the artificial dialysis equipment such as hemodialysis equipment or peritoneal dialysis equipment. For example, when used in the REDY system (recirculating dialysate system), the adsorbent of the present embodiment may be used in the adsorbent cartridge.

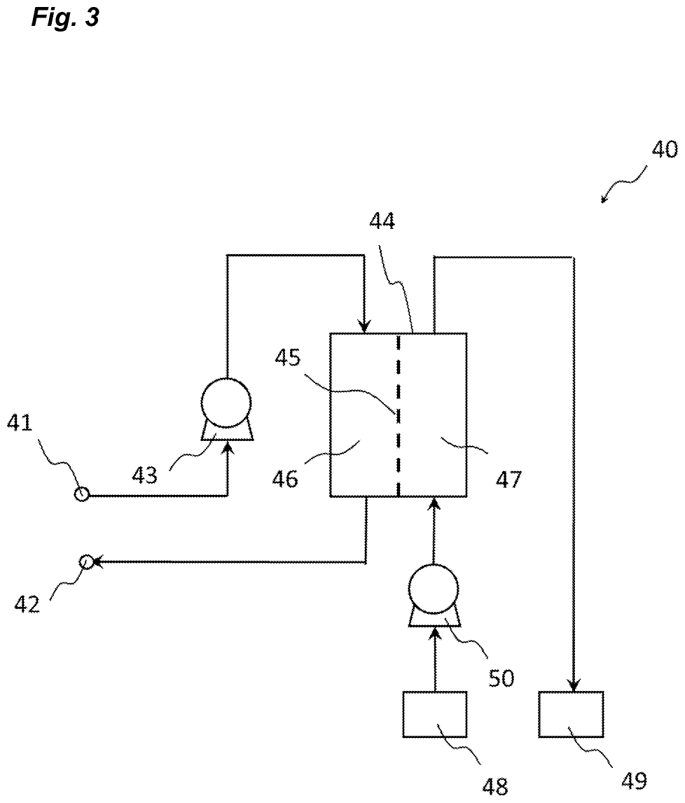

FIG. 3 schematically illustrates one-pass type hemodialysis equipment as an example of the artificial dialysis equipment using the adsorbent according to the present embodiment. In hemodialysis equipment 40 of FIG. 3, the blood before treatment introduced from a blood introduction port 41 is fed to blood purification equipment 44 by a blood pump 43. On the other hand, a dialysate is fed from an unused dialysate tank 48 to the blood purification equipment 44 by a dialysate pump 50. In the blood purification equipment 44, the blood in a blood passage area 46 of the blood purification equipment is subjected to hemodialysis, hemofiltration dialysis, or hemofiltration by a separation film 45, and the substance to be removed passes through the separation film 45 and moves to a dialysate passage area 47 of the blood purification equipment. The purified blood is sent to a blood outlet 42. On the other hand, the dialysate in the dialysate passage area 47 containing the substance to be removed is fed to a dialysate tank 49 after use. Although not illustrated in FIG. 3, a device including a path for replenishing a drug, a protein, or the like to blood as necessary may be provided before treatment and/or during delivery of blood after treatment. In addition, a sensor for measuring the blood flow rate, the dialysate flow rate, and the protein concentration in the blood as necessary may be provided. An on-off valve capable of opening and closing the flow path may be provided in the middle of the flow path of the blood and/or the dialysate as necessary.

The separation film using the adsorbent of the present embodiment is suitable for a separation film for artificial dialysis used for the hemodialysis. Examples of the material constituting the separation film other than the adsorbent generally include cellulose-based and synthetic polymer-based materials used for hemodialysis and the like. Specific examples thereof include polymethyl methacrylate, polyacrylonitrile, cellulose, cellulose acetate, polysulfone, polyvinyl alcohol, and a vinyl alcohol copolymer such as a copolymer of polyvinyl alcohol and ethylene. Polysulfone, polymethyl methacrylate, and cellulose acetate are preferably used, and polysulfone and polymethyl methacrylate are more preferably used. The form of the separation film for artificial dialysis is not particularly limited, and examples thereof include a porous type, a hollow fiber type, and a flat membrane laminated type.

As described above, the adsorbent of the present embodiment is also suitable as an adsorbent used for adsorbing a dye. Examples of the dye include methylene blue. The adsorbent is suitable, for example, for removing methylene blue, which is a dye contained in industrial water. Examples of an aspect using an adsorbent for adsorbing a dye include the above-described adsorption sheet and a separation film using the adsorbent. A material constituting the separation film used for adsorption of the dye other than the adsorbent is not particularly limited, and may be one or more materials of ceramic, metal, and a resin material. As these materials, ceramic, metal, and a resin material that can be used in the above-described composite materials can be used.

### Second Embodiment: Method for Manufacturing Adsorbent

Hereinafter, a method for manufacturing an adsorbent in the embodiment of the present invention will be described in detail, but the present invention is not limited to such an embodiment.

A method for manufacturing an adsorbent of the present embodiment comprises:

(a) preparing a precursor represented by:

MₘAXₙ
wherein M is at least one metal of Group 3, 4, 5, 6, or 7,
X is a carbon atom, a nitrogen atom, or a combination thereof,
A is at least one element of Group 12, 13, 14, 15, or 16,
n is 1 to 4, and
m is more than n and 5 or less;

(b) performing an etching treatment of removing at least a part of A atoms from the precursor by using an etching solution containing one or more of HCl, H₃PO₄, HI, and H₂SO₄ to obtain an etched product;

(c) acid-washing the etched product to obtain an acid-washed product;

(d) water-washing the acid-washed product to adjust the pH of the acid-washed product and obtain a water-washed product;

(e) performing specific metal atom intercalation treatment including a step of mixing the water-washed product with a compound containing one or more specific metal atoms selected from the group consisting of Al, Mg, Ca, Ba, Fe, Zn, Mn, or Cu to obtain a specific metal atom intercalated product; and

(f) washing the specific metal atom intercalated product with water to obtain an adsorbent. According to this manufacturing method, it is possible to manufacture an adsorbent which contains the specific metal atom and in which M in MXene is bonded to at least one selected from the group consisting of a chlorine atom, a phosphorus atom, an iodine atom, or a sulfur atom, and which is excellent in adsorption performance of, for example, a polar organic compound.

In the method for manufacturing an adsorbent of the present embodiment, in particular, the specific metal atom intercalation is performed such that etching is performed by using an etching solution containing one or more of HCl, H₃PO₄, HI, and H₂SO₄ in the etching step as described above, MXene having a large three-dimensional shape (Cl⁻, PO₄³⁻, I, and SO₄²⁻) is used on the surface, and acid-washing is performed before the intercalation of the specific metal atom to remove impurities that cause inhibition of the intercalation, whereby MXene containing the specific metal atoms between layers and having excellent adsorption performance can be easily obtained.

Hereinafter, each step of the manufacturing method will be described in detail.

Step (a)

First, a predetermined precursor is prepared. A predetermined precursor that can be used in the present embodiment is a MAX phase that is a precursor of MXene, and is represented by a formula below:

MₘAXₙ
wherein M is at least one metal of Group 3, 4, 5, 6, or 7,
X is a carbon atom, a nitrogen atom, or a combination thereof,
A is at least one element of Group 12, 13, 14, 15, or 16,
n is 1 to 4, and
m is more than n and 5 or less.

The above M, X, n, and m are as described in MXene. A is at least one element of Group 12, 13, 14, 15, or 16, is usually a Group A element, typically Group IIIA and Group IVA, more specifically, may include at least one selected from the group consisting of Al, Ga, In, Tl, Si, Ge, Sn, Pb, P, As, S, or Cd, and is preferably Al.

The MAX phase has a crystal structure in which a layer constituted by A atoms is located between two layers represented by MₘXₙ (each X may have a crystal lattice located in an octahedral array of M). Typically, in the case of m=n+1, the MAX phase has a repeating unit in which one layer of X atoms is disposed between the layers of M atoms of n+1 layers (these layers are also collectively referred to as “MₘXₙ layer”), and a layer of A atoms (“A atom layer”) is disposed as a next layer of the (n+1) th layer of M atoms; however, the present invention is not limited thereto.

The MAX phase can be produced by a known method. For example, a TiC powder, a Ti powder, and an Al powder are mixed in a ball mill, and the obtained mixed powder is calcined under an Ar atmosphere to obtain a calcined body (block-shaped MAX phase). Thereafter, the calcined body obtained is pulverized by an end mill to obtain a powdery MAX phase for the next step.

Step (b)

An etching treatment is performed to remove at least a part of A atoms from the precursor using an etching solution containing one or more of HCl, H₃PO₄, HI, and H₂SO₄. In the manufacturing method of the present embodiment, for the purpose of easily intercalating the specific metal atoms in a step (e) to be described later, in order to obtain MXene having a large three-dimensional shape (Cl⁻, PO₄³⁻, I, and SO₄²⁻) on the MXene surface, etching is performed using an etching solution containing one or more of HCl, H₃PO₄, HI, and H₂SO₄. Other conditions for the etching treatment are not particularly limited, and known conditions can be adopted. As described above, the etching can be performed using an etching solution containing F⁻, and examples thereof include a method using an etching solution further containing hydrochloric acid or the like in hydrofluoric acid, and examples of these methods include a method using a mixed solution with pure water as a solvent. Examples of the etched product obtained by the etching treatment include slurry. As the etching solution, an etching solution satisfying at least one selected from the group consisting of an HCl concentration of 6.0 M or more, an H₃PO₄ concentration of 5.5 M or more, an HI concentration of 5.0 M or more, or an H₂SO₄ concentration of 5.0 M or more can be used. In the etching of the A atoms, a part of the M atoms may be selectively etched together with the A atoms.

After the etching, water-washing can be appropriately performed. For example, stirring, centrifugation, and the like may be performed by adding water. Examples of the stirring method include stirring using a handshake, an automatic shaker, a share mixer, a pot mill, or the like. The degree of stirring such as stirring speed and stirring time may be adjusted according to the amount, concentration, and the like of the treated product which is an object to be treated. The washing with water may be performed one or more times. Preferably, washing with water is performed a plurality of times. For example, specifically, steps (i) adding water and stirring (to the etched product or the remaining precipitate obtained in the following (iii)), (ii) centrifuging the stirred product, and (iii) discarding a supernatant after centrifugation are performed within a range of 2 times or more, for example, 10 times or less.

Step (c)

The etched product obtained by the etching treatment is washed with acid.

The acid used for the acid-washing is not limited, and for example, an inorganic acid such as a mineral acid and/or an organic acid can be used. The acid is preferably only an inorganic acid or a mixed acid of an inorganic acid and an organic acid. The acid is more preferably only an inorganic acid. As the inorganic acid, for example, one or more of hydrochloric acid, sulfuric acid, nitric acid, phosphoric acid, perchloric acid, hydroiodic acid, hydrobromic acid, hydrofluoric acid, and the like can be used. It is preferably one or more of hydrochloric acid and sulfuric acid. Examples of the organic acid include acetic acid, citric acid, oxalic acid, benzoic acid, and sorbic acid. The concentration of the acid solution to be mixed with the etched product may be adjusted according to the amount, concentration, and the like of the etched product to be treated.

In the acid-washing, the etched product and the acid solution are mixed and stirred, for example. Examples of the stirring method include stirring using a handshake, an automatic shaker, a share mixer, a pot mill, or the like. The degree of stirring such as stirring speed and stirring time may be adjusted according to the amount, concentration, and the like of the etched product which is an object to be treated.

When the acid solution is mixed and stirred, heating may or may not be performed. The acid solution may be mixed and stirred without being heated, or may be stirred while being heated in a range in which the liquid temperature is 80° C. or lower.

Step (d)

The acid-washed product obtained by the acid-washing is washed with water to adjust the pH of the acid-washed product. The water-washing can be performed in the same manner as the water-washing after the etching described above. By performing the water-washing, the pH after the acid-washing is adjusted. For example, the pH of an acidic region is set to, for example, about 5 to 8. MgF₂ and CaF₂ used at the time of etching in Patent Document 1 are not preferable because they remain as insoluble compounds by adjusting the pH by water-washing.

Step (e)

The specific metal atom intercalation treatment is performed including a step of mixing the water-washed product obtained by the water-washing with a compound containing one or more specific metal atoms selected from the group consisting of Al, Mg, Ca, Ba, Fe, Zn, Mn, or Cu. As described above, the specific metal atom is larger in size than Na, K, or the like, and has an effect of widening the interlayer, so that the adsorption characteristics are improved.

As a compound containing the specific metal atom, an ionic compound in which a specific metal ion and a cation are bonded to each other can be used. Examples of the ionic compound including the specific metal ions include an iodide, a phosphate, a sulfide salt including a sulfate, a nitrate, an acetate, and a carboxylate. Compounds having low solubility such as MgF₂ and CaF₂ as described above are not included.

The content of a compound containing the specific metal atom in a formulation for intercalation treatment is preferably 0.001 mass % or more. The content is more preferably 0.01 mass % or more, and still more preferably 0.1 mass % or more. On the other hand, from the viewpoint of dispersibility in a solution, the content of the compound containing the specific metal atom is preferably 10 mass % or less, and more preferably 1 mass % or less.

The specific method of intercalation treatment is not particularly limited, and for example, the compound containing the specific metal atom may be mixed with the moisture medium clay of the MXene and stirred, or may be allowed to stand. For example, stirring at room temperature can be mentioned. Examples of the stirring method include a method using a stirring bar such as a stirrer, a method using a stirring blade, a method using a mixer, and a method using a centrifugal device, and the stirring time can be set according to the manufacturing scale of the adsorbent, and for example, the stirring time is set to 12 to 24 hours.

Step (f)

The specific metal atom intercalated product obtained by performing the specific metal atom intercalation treatment is washed with water to obtain an adsorbent. The water-washing can be performed in the same manner as the water-washing after the etching described above. For example, a step of centrifuging the slurry-like specific metal atom intercalated product to discard the supernatant and then washing the remaining precipitate with water is repeated, and for example, clay-like MXene in which the specific metal atoms are intercalated can be obtained.

According to the manufacturing method of the present embodiment, protons derived from the acidic substance used in the etching and the acid-washing and remaining between the layers are discharged and removed to the outside of the layer during the intercalation treatment of the specific metal atoms and the water-washing in the step (f), and thus the resulting adsorbent does not cause a decrease in pH of the solution and is excellent in pH stability even when the adsorbent is subsequently immersed in a solution.

Although the adsorbent and the manufacturing method thereof, the adsorption sheet, the separation film, and the artificial dialysis equipment in the embodiment of the present invention have been described in detail above, various modifications can be made. It should be noted that the adsorbent according to the present invention may be produced by a method different from the manufacturing method in the above-described embodiment, and the method for manufacturing an adsorbent according to the present invention is not limited only to one that provides the adsorbent according to the above-described embodiment.

### EXAMPLES

[Preparation of MXene Adsorbent]

In the present examples, steps of (1) preparation of the precursor (MAX), (2) etching of the precursor, (3) water-washing after etching, (4) acid-washing (removal of Al residues derived from MAX), (5) water-washing after acid-washing, (6) specific metal atom intercalation, (7) washing after intercalation, and (8) freeze-drying described in detail below were sequentially performed to prepare an adsorbent formed of the specific metal atom-containing MXene.

(1) Preparation of Precursor (MAX)

TiC powder, Ti powder, and Al powder (all manufactured by Kojundo Chemical Laboratory Co., Ltd.) were placed in a ball mill containing zirconia balls at a molar ratio of 2:1:1 and mixed for 24 hours. The obtained mixed powder was calcined at 1350° C. for 2 hours under an Ar atmosphere. The calcined body (block-shaped MAX) thus obtained was pulverized with an end mill to a maximum dimension of 40 m or less. In this way, Ti₃AlC₂ particles were obtained as a precursor (powdery MAX).

(2) Etching of Precursor

Using the Ti₃AlC₂ particles (powder) prepared by the above method, etching was performed under the following etching conditions to obtain a solid-liquid mixture (slurry) containing a solid component derived from the Ti₃AlC₂ powder. In the present example, it is considered that a chlorine atom is bonded to M of the layer of MXene derived from hydrochloric acid (HCl) contained in the etching solution used in this etching.

(Etching Conditions)
Precursor: Ti₃AlC₂ (sieving with a mesh size of 45 μm)
Etching solution composition: 49% HF 6 mL,
   H₂O 18 mL
   HCl (12M) 36 mL
Amount of precursor input: 3.0 g
Etching container: 100 mL Aiboy
Etching temperature: 35° C.
Etching time: 24 h
Stirrer rotation speed: 400 rpm

(3) Water-Washing after Etching

The slurry was equally divided into three portions, each of which was inserted into three 50 mL centrifuge tubes, centrifuged under the condition of 3,500 G using a centrifuge, and then the supernatant was discarded. An operation of adding 40 mL of pure water to the remaining precipitate in each centrifuge tube, centrifuging again at 3,500 G, and separating and removing the supernatant was repeated 11 times to obtain a slurry as a water-washed product.

(4) Acid-Washing (Removal of Al Residues Derived from MAX)

40 mL of 1 M hydrochloric acid was added to the above slurry, then the mixture was stirred with a shaker for 5 minutes, then centrifuged at 3,500 G, and the supernatant was discarded.

(5) Water-Washing after Acid-Washing

(i) 40 mL of pure water was added to the remaining precipitate in each centrifuge tube and (ii) centrifuged at 3,500 G to (iii) separate and remove the supernatant. The operations (i) to (iii) were repeated 5 times in total. After final centrifugation, the supernatant was discarded to obtain a Ti₃C₂Tₛ-moisture medium clay.

(6) Intercalation of Specific Metal Atoms (Mg or Ca or Al)

The Ti₃C₂Tₛ-moisture medium clay prepared by the above method was intercalated with the specific metal atom (Mg, Ca, or Al) using each intercalator shown in Table 1. The detailed conditions of intercalation are as follows. In the following conditions, the stirring time is set to 18 hours, but the stirring time can be set according to the manufacturing scale of the MXene adsorbent, and can be set to, for example, 12 to 24 hours.

(Conditions of Intercalation of Mg, Ca, or Al)
Ti₃C₂Tₛ-moisture medium clay (MXene after washing): Solid content 1.0 g
MgCl₂: 2.34 g (Example 1), or CaCl₂: 3.16 g (Example 2), or AlCl₃: 3.15 g Example 3
Pure water: 20 mL
Intercalation container: 100 mL Aiboy
(Stirring) Temperature: 20° C. or higher and 25° C. or lower (room temperature)
(Stirring) Time: 18 hours
Stirrer rotation speed: 800 rpm

(7) Water-Washing after Intercalation of Mg, Ca, or Al

Each of the slurries obtained by intercalation with Mg, Ca, or Al was transferred to a centrifuge tube, (i) 40 mL of pure water was added, (ii) centrifugation was performed under the condition of 3,500 G using a centrifuge, and (iii) a supernatant was separated and removed. The operations (i) to (iii) were repeated five times in total to remove excess Mg, Ca, or Al, thereby obtaining each MXene clay intercalated with Ma, Ca, or Al. A filtration film (MXene film) used for XRD measurement to be described later was obtained by suction filtration using the MXene clay. After the filtration, vacuum drying was performed at 80° C. for 24 hours to prepare an MXene film. As a filter for suction filtration, a membrane filter (Durapore, manufactured by Merck KGaA, pore size 0.45 m) was used.

(8) Drying

Each of the MXene clays was frozen at −40° C. for 5 hours, and then dried in a freeze dryer for 24 hours to obtain MXene dry powder of Example 1, Example 2, and Example 3. This dry powder was used as an adsorbent for MXene.

As comparative examples, an adsorbent of Comparative Example 1 manufactured in the same manner as described above except for using Na, an adsorbent of Comparative Example 2 manufactured in the same manner as described above except for using K, and an adsorbent of Comparative Example 3 manufactured by the method disclosed in Non-patent Document 1, that is, manufactured without using hydrochloric acid for etching and without performing intercalation were also prepared.

[Evaluation of MXene Adsorbent]

[Evaluation of Interlayer Distance]

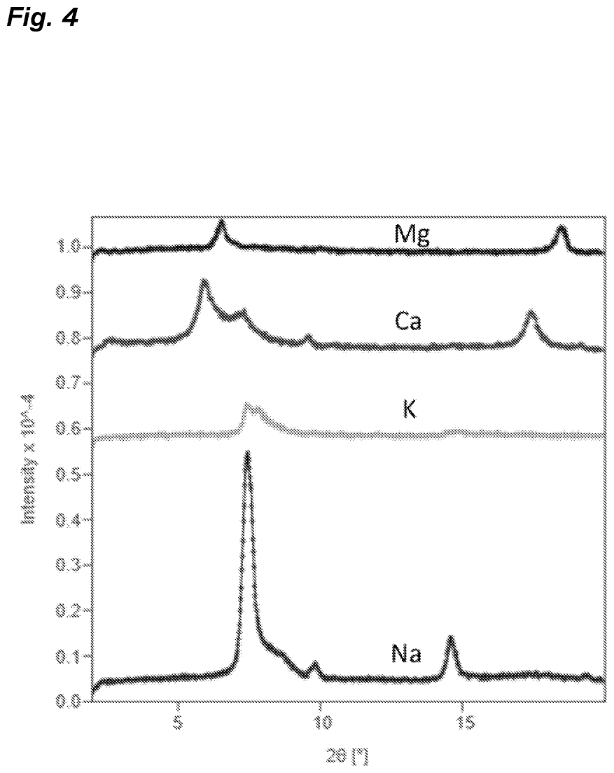

The interlayer distance of MXene constituting the adsorbent was measured. More specifically, XRD measurement of the adsorbents of Examples 1 to 3 and Comparative Examples 1 and 2 was performed under the following conditions to obtain two-dimensional X-ray diffraction images of the MXene film. The results of Examples 1 and 2 and Comparative Examples 1 and 2 are shown in FIG. 4.

(XRD Measurement Conditions)
Equipment used: MiniFlex 600 manufactured by Rigaku Corporation
Conditions
   Light source: Cu tube bulb
   Characteristic X-ray: CuKα=1.54 Å
   Measurement range: 3 degrees to 20 degrees
   Step: 50 step/degree

As a result of calculating the interlayer distance from the XRD measurement result, it was 13.5 Å in Example 1, 14.9 Å in Example 2, and 13.0 Å in Example 3. In Comparative Example 1, it was 11.8 Å, and in Comparative Example 2, it was also 11.8 Å.

From FIG. 4 and the calculation results of the interlayer distance, in Examples 1 to 3, since intercalation of Mg, Ca, and Al was performed, the peak of the (002) plane was on the low angle side, and the interlayer was widened. On the other hand, in Comparative Examples 1 and 2, intercalations of Na and K were respectively performed, but since these atoms were smaller in size than Mg and Ca, the interlayer distance did not sufficiently increase.

[Measurement of Content of Specific Metal Atom (Mg, Ca, Al) in MXene] MXene was made into a solution by an alkali melting method, and the Mg content in MXene of Example 1, the Ca content in MXene of Example 2, and the Al content in MXene of Example 3 (all corresponding to the residual amount of an intercalator) were measured by ICP-AES (iCAP 7400 manufactured by Thermo Fisher Scientific was used) using inductively coupled plasma emission spectrometry. As a result, the Mg content was 0.78 mass % in Example 1, the Ca content was 1.37 mass % in Example 2, and the Al content was 0.58 wt % in Example 3. In any of the examples, it was also separately checked that the Li content was equal to or less than the quantification limit, that is, 0.0001 mass % or less.

[Evaluation on Amount of Acidic Substance Between Layers]

The pH when the adsorbent was immersed in pure water was measured, and the presence or absence of outflow of an acidic substance that can be inserted between layers in the production process was evaluated. As a result, in the case of Comparative Example 3 without an intercalator, the pH of the immersed pure product was 3.59, whereas the pH of Example 1 (intercalator: Mg) was 5.34, and the pH of Example 2 (intercalator: Ca) was 5.15. The pH of Example 3 (intercalator: Al) was 5.12. From these results, in the case of Comparative Example 3 without an intercalator, the acidic substance inserted between the layers in the process of manufacturing the adsorbent flowed out after manufacturing the adsorbent and showed strong acidity, whereas in the case of Examples 1 to 3, the acidic substance used in the manufacturing process was considered to be easily removed at the time of manufacturing since the layers between the adsorbents were wide, and as a result, a decrease in pH when the adsorbent was immersed in pure water could be suppressed.

[Evaluation of Adsorption Performance]

Using the adsorbents of Examples 1 to 3 and Comparative Examples 1 to 3, the adsorption amount of the substance to be adsorbed (urea) was measured as follows, and the adsorption performance of the adsorbent was evaluated.

(1) Preparation of Urea Solution

0.5 g of urea was weighed, added to 100 mL of pure water, and diluted 100 times to prepare a urea solution having a concentration of 5 mg/dL.

(2) Preparation of Assay Kit Solution

Using a bioassay kit (product name: DIUR-100) manufactured by Funakoshi Co., Ltd., liquid A and liquid B of the kit were mixed in equal volume to prepare an assay kit solution.

(3) Preparation of Solution Containing Substance to be Adsorbed (Urea Solution)

250 mL of the urea solution prepared in the procedure (1) was put into a 500 mL beaker, and heated and stirred at a rotation speed of 400 rpm and a liquid temperature of 37° C. using a hot stirrer to prepare a solution containing urea as a substance to be adsorbed. Six urea solutions were prepared for respective examples.

(4) Urea Adsorption, Sample Sampling

0.1 g of each of the adsorbents of Examples 1 to 3 and Comparative Examples 1 to 3 was added to the urea solution prepared in the procedure (3), and stirred with a hot stirrer for 30 minutes. Thereafter, the solutions after standing were each collected with a 10 mL pipette, the floating adsorbent was precipitated and separated by a centrifuge under the conditions of 20,000 rpm and 10 minutes, and 250 μL of the supernatant was sampled.

(5) Dropwise Addition of Assay Kit Solution

1,250 μL of the assay kit solution prepared in the procedure (2) was added to the supernatant, and the mixture was allowed to stand for 50 minutes.

(6) Absorbance Measurement

First, for creating a calibration curve, a urea solution to which an adsorbent was not added and a solution obtained by diluting the urea solution to which the adsorbent was not added twice were prepared. Then, the absorbance of each solution was measured to create a calibration curve. Next, the absorbance of the sample prepared in the procedure (5) was measured, each absorbance was compared with a calibration curve to determine the concentration of urea remaining without being adsorbed in the solution, and the urea adsorption amount (urea amount (mg) per 1 g of the adsorbent) was calculated from the concentration of the urea. The results are shown in Table 1.

TABLE 1

| | Presence or | | Urea |
|---|---|---|---|
| | absence of use | Metal | adsorption |
| | of hydrochloric | elements for | amount |
| | acid during etching | intercalator | (mg/g) |
| Example 1 | Presence | Mg | 17 |
| Example 2 | Presence | Ca | 17 |
| Example 3 | Presence | Al | 16 |
| Comparative | Presence | Na | 3 |
| Example 1 | | | |
| Comparative | Presence | K | 5 |
| Example 2 | | | |
| Comparative | Absence | Absence | 9.7 |
| Example 3 | | | |

From the above results, it is considered that when Na and K were used, the interlayer distance was small, and Na and K existed at a molecular adsorption site, resulting in inhibition of adsorption, and therefore the adsorption performance was lower than that of Comparative Example 3 without an intercalator. On the other hand, in Examples 1, 2, and 3 in which Mg, Ca, and Al were respectively used as intercalators, it is considered that a structure of MXene having an interlayer distance suitable for the urea molecular size was obtained, and thus high adsorption performance of urea was exhibited.

In each of Examples 1, 2, and 3, Mg²⁺, Ca²⁺, and Al³⁺ are intercalated between MXene layers, but Li is not used in the manufacturing process of the adsorbent, and thus Li is not contained. In the techniques of Non-patent Document 1 and Non-patent Document 2, it is difficult to sufficiently reduce the Li content, but according to the adsorbent of the present embodiment, it is also possible to cope with applications in which Li needs to be reduced as much as possible. Furthermore, as disclosed in Patent Document 1, when MgF₂ and CaF₂ are used during etching, these compounds have low solubility, and thus can remain as impurities in the material. Therefore, for example, when the compound is not allowed to remain, it is considered that further improvement is necessary, but according to the adsorbent of the present embodiment, hardly soluble impurities such as MgF₂ and CaF₂ are not contained. Therefore, in particular, an adsorbent using Mg or Ca as an intercalator is excellent in biocompatibility. Furthermore, as described above, since the adsorbent of Examples 1 to 3 does not contain much acidic substances used in manufacturing and the like, a decrease in the pH of the solution when the adsorbent is immersed in the solution is suppressed, and thus, the adsorbent is also excellent in pH stability.

[Evaluation of Adsorption Performance of Dye (Methylene Blue)]

Using MXene of Examples 1 to 3 and Comparative Examples 1 to 3 described above, the substance to be adsorbed was methylene blue as an example of a dye, and adsorption evaluation was performed.

(1) Preparation of Methylene Blue Solution

0.1 g of methylene blue was weighed and added to 2 L of pure water to prepare a urea solution having a concentration of 5 mg/L.

(2) Preparation of Solution Containing Substance to be Adsorbed (Urea Solution)

250 mL of the methylene blue solution prepared in the procedure (7) was put into a 500 mL beaker, and heated and stirred at a rotation speed of 400 rpm and a liquid temperature of 20° C. using a stirrer to prepare a solution containing methylene blue as a substance to be adsorbed. Six urea solutions were prepared for respective examples.

(3) Urea Adsorption, Sample Sampling

0.01 g of each of the adsorbents of Examples 1 to 3 and Comparative Examples 1 to 3 was added to the methylene blue solution prepared in the procedure (8), and stirred with a stirrer for 30 minutes. Thereafter, the solutions after standing were each collected with a 10 mL pipette, the floating adsorbent was precipitated and separated by a centrifuge under the conditions of 3,500 G and 5 minutes, and 1,000 μL of the supernatant was sampled.

(4) Absorbance Measurement

First, for creating a calibration curve, a methylene blue solution to which an adsorbent was not added and a solution obtained by diluting the methylene blue solution to which the adsorbent was not added twice were prepared. Then, the absorbance of each solution was measured to create a calibration curve. Next, the absorbance of the sample prepared in the procedure (9) was measured, each absorbance was compared with a calibration curve to determine the concentration of methylene blue remaining without being adsorbed in the solution, and the methylene blue adsorption amount (methylene blue amount (mg) per 1 g of the adsorbent) was calculated from the concentration of the methylene blue. The results are shown in Table 2.

TABLE 2

| | Presence or | | Methylene blue |
|---|---|---|---|
| | absence of use | Metal | adsorption |
| | of hydrochloric | elements for | amount |
| | acid during etching | intercalator | (mg/g) |
| Example 1 | Presence | Mg | 150 |
| Example 2 | Presence | Ca | 121 |
| Example 3 | Presence | Al | 114 |
| Comparative | Presence | Na | 36 |
| Example 1 | | | |
| Comparative | Presence | K | 35 |
| Example 2 | | | |
| Comparative | Absence | Absence | 37 |
| Example 3 | | | |

From the above results, it is considered that when Na an K were used, the interlayer distance was small, and Na and K existed at a molecular adsorption site, resulting in inhibition of adsorption, and therefore the adsorption performance was lower than that of Comparative Example 3 without an intercalator. On the other hand, in Examples 1, 2, and 3 in which Mg, Ca, and Al were respectively used as intercalators, it is considered that a structure of MXene having an interlayer distance suitable for the methylene blue molecular size was obtained, and thus high adsorption performance was exhibited.

The adsorbent of the present invention can be used in any suitable application, and can be preferably used, for example, as a separation film in an artificial dialysis equipment and the like.

### EXPLANATION OF REFERENCES

1a, 1b Layer body (MₘXₙ layer)
3a, 5a, 3b, 5b Modifier or terminal T
7a, 7b MXene layer
10a, 10b MXene particles (particles of layered material)
20 Titanium atom
21 Oxygen atom
40 Hemodialysis equipment
41 Blood inlet
42 Blood outlet
43 Blood pump
44 Blood purification equipment
45 Separation film
46 Blood passage area of blood purification equipment
47 Dialysate passage area of blood purification equipment
48 Unused dialysate tank
49 Post-use dialysate tank
50 Dialysate pump

1. An adsorbent comprising:
particles of a layered material including one or plural layers; and
one or more metal atoms selected from the group consisting of Al, Mg, Ca, Ba, Mn, or Cu,
wherein the one or plural layers include a layer body represented by:
MₘXₙ
wherein M is at least one metal of Group 3, 4, 5, 6, or 7,
X is a carbon atom, a nitrogen atom, or a combination thereof,
n is 1 to 4, and
m is more than n and 5 or less, and
a modifier or terminal T exists on a surface of the layer body, wherein T is at least one selected from the group consisting of a hydroxyl group, a fluorine atom, a chlorine atom, an oxygen atom, or a hydrogen atom, and
wherein the M of the layer body is bonded to at least one selected from the group consisting of a chlorine atom, a phosphorus atom, an iodine atom, or a sulfur atom.

2. The adsorbent according to claim 1, wherein the particles of the layered material include the plural layers.

3. The adsorbent according to claim 1, wherein the M of the layer body is bonded to at least one selected from the group consisting of Cl⁻, PO₄³⁻, I, or SO₄²⁻.

4. The adsorbent according to claim 1, wherein the one or more metal atoms are one or more selected from the group consisting of Mg, Ca, or Mn.

5. The adsorbent according to claim 1, wherein the one or more metal atoms are Mg or Ca.

6. The adsorbent according to claim 1, wherein a total content of the Mg and the Ca in the one or more metal atoms is 0.001 mass % to 1.5 mass %.

7. The adsorbent according to claim 1, wherein a Li content is 0.0001 mass % or less.

8. The adsorbent according to claim 1, further comprising one or more materials selected from a ceramic, a metal, and a resin.

9. The adsorbent according to claim 1, wherein the adsorbent is in a sheet-like form.

10. The adsorbent according to claim 1, wherein the adsorbent is constructed to adsorb a polar organic compound.

11. The adsorbent according to claim 1, wherein the adsorbent is constructed to adsorb a compound having one or more of a hydroxyl group and an amino group, and ammonia.

12. An adsorbent used for adsorbing uremic toxin, the adsorbent comprising:
particles of a layered material including one or plural layers; and
one or more metal atoms selected from the group consisting of Al, Mg, Ca, Ba, Fe, Zn, Mn, or Cu,
wherein the one or plural layers include a layer body represented by:
MₘXₙ
wherein M is at least one metal of Group 3, 4, 5, 6, or 7,
X is a carbon atom, a nitrogen atom, or a combination thereof,
n is 1 to 4, and
m is more than n and 5 or less, and
a modifier or terminal T exists on a surface of the layer body, wherein T is at least one selected from the group consisting of a hydroxyl group, a fluorine atom, a chlorine atom, an oxygen atom, or a hydrogen atom, and
wherein the M of the layer body is bonded to at least one selected from the group consisting of a chlorine atom, a phosphorus atom, an iodine atom, or a sulfur atom.

13. An adsorbent used for adsorbing urea, the adsorbent comprising:
particles of a layered material including one or plural layers; and
one or more metal atoms selected from the group consisting of Al, Mg, Ca, Ba, Fe, Zn, Mn, or Cu,
wherein the one or plural layers include a layer body represented by:
MₘXₙ
wherein M is at least one metal of Group 3, 4, 5, 6, or 7,
X is a carbon atom, a nitrogen atom, or a combination thereof,
n is 1 to 4, and
m is more than n and 5 or less, and
a modifier or terminal T exists on a surface of the layer body, wherein T is at least one selected from the group consisting of a hydroxyl group, a fluorine atom, a chlorine atom, an oxygen atom, or a hydrogen atom, and
wherein the M of the layer body is bonded to at least one selected from the group consisting of a chlorine atom, a phosphorus atom, an iodine atom, or a sulfur atom.

14. The adsorbent according to claim 1, wherein the adsorbent is constructed to adsorb a dye.

15. The adsorbent according to claim 14, wherein the dye is methylene blue.

16. An adsorption sheet comprising the adsorbent according to claim 1.

17. A separation film comprising the adsorbent according to claim 1.

18. An artificial dialysis equipment comprising an adsorbent, the adsorbent comprising:
particles of a layered material including one or plural layers; and
one or more metal atoms selected from the group consisting of Al, Mg, Ca, Ba, Fe, Zn, Mn, or Cu,
wherein the one or plural layers include a layer body represented by:
MₘXₙ
wherein M is at least one metal of Group 3, 4, 5, 6, or 7,
X is a carbon atom, a nitrogen atom, or a combination thereof,
n is 1 to 4, and
m is more than n and 5 or less, and
a modifier or terminal T exists on a surface of the layer body, wherein T is at least one selected from the group consisting of a hydroxyl group, a fluorine atom, a chlorine atom, an oxygen atom, or a hydrogen atom, and
wherein the M of the layer body is bonded to at least one selected from the group consisting of a chlorine atom, a phosphorus atom, an iodine atom, or a sulfur atom.

19. A method for manufacturing an adsorbent, the method comprising:
(a) preparing a precursor represented by a formula below:
MₘAXₙ
wherein M is at least one metal of Group 3, 4, 5, 6, or 7,
X is a carbon atom, a nitrogen atom, or a combination thereof,
A is at least one element of Group 12, 13, 14, 15, or 16,
n is 1 to 4, and
m is more than n and 5 or less;
(b) performing etching treatment of removing at least a part of A atoms from the precursor by using an etching solution containing one or more of HCl, H₃PO₄, HI, and H₂SO₄ to obtain an etched product;
(c) acid-washing the etched product to obtain an acid-washed product;
(d) water-washing the acid-washed product to adjust the pH of the acid-washed product and obtain a water-washed product;
(e) performing metal atom intercalation treatment including a step of mixing the water-washed product with a compound containing one or more metal atoms selected from the group consisting of Al, Mg, Ca, Ba, Mn, or Cu to obtain a metal atom intercalated product; and
(f) washing the metal atom intercalated product with water to obtain an adsorbent.
